(19) United States
(12) Patent Application Publication
(10) Pub. No.: US 2014/0180139 A1
(43) Pub. Date: Jun. 26, 2014

(54) Connectors for Use With Intravascular Devices and Associated Systems and Methods

(71) Applicant: Volcano Corporation, San Diego, CA (US)

(72) Inventors: Bret C. Millett, Folsom, CA (US); Ky Huynh, Tigard, OR (US); Lonnie Milton, Mulino, OR (US)

(73) Assignee: Volcano Corporation, San Diego, CA (US)

(21) Appl. No.: 14/133,363

(22) Filed: Dec. 18, 2013

Related U.S. Application Data
(60) Provisional application No. 61/745,472, filed on Dec. 21, 2012.

(51) Int. Cl. A61B 5/00; A61B 5/026; A61B 5/0215

(52) U.S. Cl. CPC A61B 5/6852; A61B 5/0215; A61B 5/026; A61B 5/6851
USPC 600/486; 600/505

(57) ABSTRACT

Intravascular devices, systems, and methods are disclosed. In some embodiments, side-loading electrical connectors for use with intravascular devices are provided. The side-loading electrical connector has at least one electrical contact configured to interface with an electrical connector of the intravascular device. A first connection piece of the side-loading electrical connector is movable relative to a second connection piece between an open position and a closed position, wherein in the open position an elongated opening is formed between the first and second connection pieces to facilitate insertion of the electrical connector between the first and second connection pieces in a direction transverse to a longitudinal axis of the intravascular device and wherein in the closed position the at least one electrical contact is electrically coupled to the at least one electrical connector received between the first and second connection pieces.

### CROSS-REFERENCE TO RELATED APPLICATIONS

The present application claims priority to and the benefit of U.S. Provisional Patent Application No. 61/745,472, filed Dec. 21, 2012, which is hereby incorporated by reference in its entirety.

### TECHNICAL FIELD

The present disclosure relates to intravascular devices, systems, and methods. In some embodiments, the intravascular devices are guidewires that include one or more electronic components.

### BACKGROUND

Heart disease is very serious and often requires emergency operations to save lives. A main cause of heart disease is the accumulation of plaque inside the blood vessels, which eventually occludes the blood vessels. Common treatment options available to open up the occluded vessel include balloon angioplasty, rotational atherectomy, and intravascular stents. Traditionally, surgeons have relied on X-ray fluoroscopic images that are planar images showing the external shape of the silhouette of the lumen of blood vessels to guide treatment. Unfortunately, with X-ray fluoroscopic images, there is a great deal of uncertainty about the exact extent and orientation of the stenosis responsible for the occlusion, making it difficult to find the exact location of the stenosis. In addition, though it is known that restenosis can occur at the same place, it is difficult to check the condition inside the vessels after surgery with X-ray.

A currently accepted technique for assessing the severity of a stenosis in a blood vessel, including ischemia causing lesions, is fractional flow reserve (FFR). FFR is a calculation of the ratio of a distal pressure measurement (taken on the distal side of the stenosis) relative to a proximal pressure measurement (taken on the proximal side of the stenosis). FFR provides an index of stenosis severity that allows determination as to whether the blockage limits blood flow within the vessel to an extent that treatment is required. The normal value of FFR in a healthy vessel is 1.00, while values less than about 0.80 are generally deemed significant and require treatment.

Often intravascular catheters and guidewires are utilized to measure the pressure within the blood vessel. To date, guidewires containing pressure sensors or other electronic components have suffered from reduced performance characteristics compared to standard guidewires that do not contain electronic components. For example, the handling performance of previous guidewires containing electronic components have been hampered, in some instances, by the limited space available for the core wire after accounting for the space needed for the conductors or communication lines of the electronic component(s), the stiffness of the rigid housing containing the electronic component(s), and/or other limitations associated with providing the functionality of the electronic components in the limited space available within a guidewire. Further, due to its small diameter, in many instances the proximal connector portion of the guidewire (i.e., the connector(s) that facilitate communication between the electronic component(s) of the guidewire and an associated controller or processor) is fragile and prone to kinking, which destroys the functionality of the guidewire. For this reason, surgeons are reluctant to remove the proximal connector from the guidewire during a procedure for fear of breaking the guidewire when reattaching the proximal connector. However, having the guidewire coupled to the proximal connector further limits the maneuverability and handling of the guidewire.

Accordingly, there remains a need for improved connectors for use with intravascular devices (e.g., catheters and guidewires) that include one or more electronic components.

### SUMMARY

Embodiments of the present disclosure are directed to intravascular devices, systems, and methods.

In some embodiments, side-loading electrical connectors for use with intravascular devices are provided. The side-loading electrical connector has at least one electrical contact configured to interface with an electrical connector of the intravascular device. A first connection piece of the side-loading electrical connector is movable relative to a second connection piece between an open position and a closed position, wherein in the open position an elongated opening is formed between the first and second connection pieces to facilitate insertion of the electrical connector between the first and second connection pieces in a direction transverse to a longitudinal axis of the intravascular device and wherein in the closed position the at least one electrical contact is electrically coupled to the at least one electrical connector received between the first and second connection pieces.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic side view of an intravascular device according to an embodiment of the present disclosure.

FIG. 2 is a diagrammatic schematic view of an intravascular system according to an embodiment of the present disclosure.

FIG. 3 is a diagrammatic perspective view of a connector according to an embodiment of the present disclosure.

FIG. 4 is a diagrammatic perspective view of a portion of the connector of FIG. 4 and a proximal connector portion of an intravascular device according to an embodiment of the present disclosure.

FIG. 5 is a diagrammatic, partial cross-sectional side view of a connector having an opening in communication with a central lumen according to an embodiment of the present disclosure.

FIG. 6 is a diagrammatic, partial cross-sectional side view of a connector having an opening in communication with a central lumen according to another embodiment of the present disclosure.

FIG. 7 is a diagrammatic cutaway end view of a connector in an open position according to an embodiment of the present disclosure.

FIG. 8 is a diagrammatic cutaway side view of the connector of FIG. 7 in the open position according to an embodiment of the present disclosure.

FIG. 9 is a diagrammatic cutaway end view of the connector of FIGS. 7 and 8 in a closed position according to an embodiment of the present disclosure.

FIG. 10 is a diagrammatic cutaway side view of the connector of FIGS. 7-9 in the closed position according to an embodiment of the present disclosure.

FIG. 11 is a diagrammatic top view of a contact forming structure according to an embodiment of the present disclosure.

FIG. 12 is a diagrammatic top view of the contact forming structure of FIG. 11 in a subsequent step of manufacturing according to an embodiment of the present disclosure.

FIG. 13 is a diagrammatic top view of a mounting structure with a plurality of electrical contacts secured thereto according to an embodiment of the present disclosure.

FIG. 14 is a diagrammatic end view of a mounting structure and associated electrical contact in an open position, spaced from a connection portion of an intravascular device according to an embodiment of the present disclosure.

FIG. 15 is a diagrammatic end view of the mounting structure and associated electrical contact of FIG. 14 in a closed position, engaged with the connection portion of the intravascular device according to an embodiment of the present disclosure.

FIG. 16 is a diagrammatic perspective view of an electrical contact according to an embodiment of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

As used herein, “flexible elongate member” or “elongate flexible member” includes at least any thin, long, flexible structure that can be inserted into the vasculature of a patient. While the illustrated embodiments of the “flexible elongate members” of the present disclosure have a cylindrical profile with a circular cross-sectional profile that defines an outer diameter of the flexible elongate member, in other instances all or a portion of the flexible elongate members may have other geometric cross-sectional profiles (e.g., oval, rectangular, square, elliptical, etc.) or non-geometric cross-sectional profiles. Flexible elongate members include, for example, intravascular catheters and intravascular guidewires. In that regard, intravascular catheters may or may not include a lumen extending along its length for receiving and/or guiding other instruments. If the intravascular catheter includes a lumen, the lumen may be centered or offset with respect to the cross-sectional profile of the device.

In most embodiments, the flexible elongate members of the present disclosure include one or more electronic, optical, or electro-optical components. For example, without limitation, a flexible elongate member may include one or more of the following types of components: a pressure sensor, a temperature sensor, an imaging element, an optical fiber, an ultrasound transducer, a reflector, a minor, a prism, an ablation element, an fro electrode, a conductor, and/or combinations thereof. Generally, these components are configured to obtain data related to a vessel or other portion of the anatomy in which the flexible elongate member is disposed. Often the components are also configured to communicate the data to an external device for processing and/or display. In some aspects, embodiments of the present disclosure include imaging devices for imaging within the lumen of a vessel, including both medical and non-medical applications. However, some embodiments of the present disclosure are particularly suited for use in the context of human vasculature. Imaging of the intravascular space, particularly the interior walls of human vasculature can be accomplished by a number of different techniques, including ultrasound (often referred to as intravascular ultrasound (“IVUS”) and intracardiac echocardiography (“ICE”)) and optical coherence tomography (“OCT”). In other instances, infrared, thermal, or other imaging modalities are utilized. Further, in some instances the flexible elongate member includes multiple electronic, optical, and/or electro-optical components (e.g., pressure sensors, temperature sensors, imaging elements, optical fibers, ultrasound transducers, reflectors, mirrors, prisms, ablation elements, fro electrodes, conductors, etc.).

The electronic, optical, and/or electro-optical components of the present disclosure are often disposed within a distal portion of the flexible elongate member. As used herein, “distal portion” of the flexible elongate member includes any portion of the flexible elongate member from the mid-point to the distal tip. As flexible elongate members can be solid, some embodiments of the present disclosure will include a housing portion at the distal portion for receiving the electronic components. Such housing portions can be tubular structures attached to the distal portion of the elongate member. Some flexible elongate members are tubular and have one or more lumens in which the electronic components can be positioned within the distal portion.

The electronic, optical, and/or electro-optical components and the associated communication lines are sized and shaped to allow for the diameter of the flexible elongate member to be very small. For example, the outside diameter of the elongate member, such as a guidewire or catheter, containing one or more electronic, optical, and/or electro-optical components as described herein are between about 0.0007″ (0.0178 mm) and about 0.118″ (3.0 mm), with some particular embodiments having outer diameters of approximately 0.014″ (0.3556 mm) and approximately 0.018″ (0.4572 mm)). As such, the flexible elongate members incorporating the electronic, optical, and/or electro-optical component(s) of the present application are suitable for use in a wide variety of lumens within a human patient besides those that are part or immediately surround the heart, including veins and arteries of the extremities, renal arteries, blood vessels in and around the brain, and other lumens.

“Connected” and variations thereof as used herein includes direct connections, such as being glued or otherwise fastened directly to, on, within, etc. another element, as well as indirect connections where one or more elements are disposed between the connected elements.

“Secured” and variations thereof as used herein includes methods by which an element is directly secured to another element, such as being glued or otherwise fastened directly to, on, within, etc. another element, as well as indirect techniques of securing two elements together where one or more elements are disposed between the secured elements.

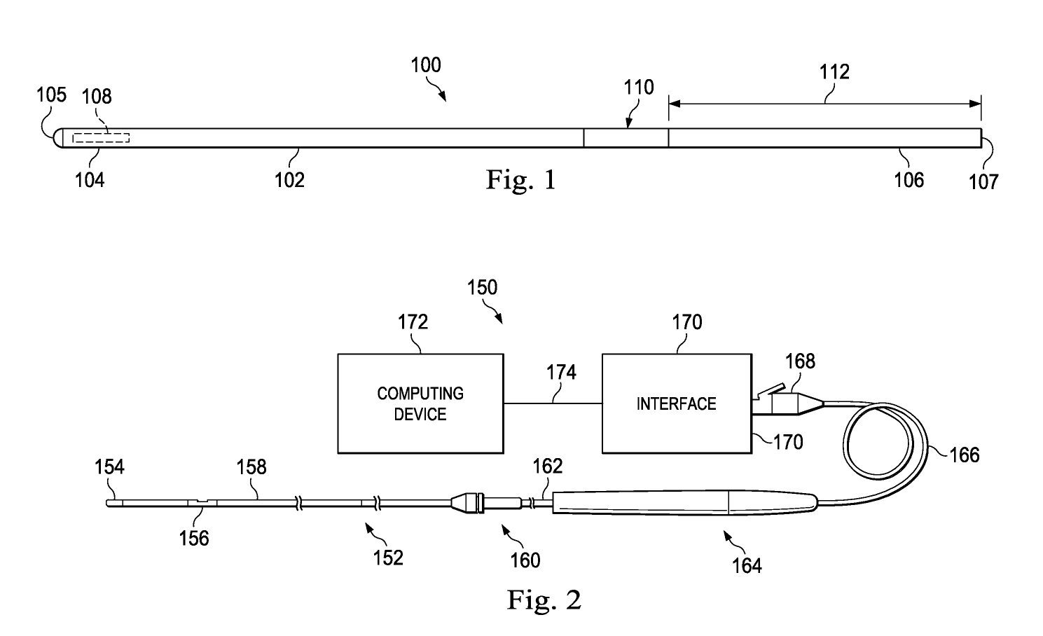

Referring now to FIG. 1, shown therein is a portion of an intravascular device 100 according to an embodiment of the present disclosure. In that regard, the intravascular device 100 includes a flexible elongate member 102 having a distal portion 104 adjacent a distal end 105 and a proximal portion 106 adjacent a proximal end 107. A component 108 is positioned within the distal portion 104 of the flexible elongate member 102 proximal of the distal tip 105. Generally, the component 108 is representative of one or more electronic, optical, or electro-optical components. In that regard, the component 108 is a pressure sensor, a temperature sensor, an imaging element, an optical fiber, an ultrasound transducer, a reflector, a minor, a prism, an ablation element, an RF electrode, a conductor, and/or combinations thereof. The specific type of component or combination of components can be selected based on an intended use of the intravascular device. In some instances, the component 108 is positioned less than 10 cm, less than 5, or less than 3 cm from the distal tip 105. In some instances, the component 108 is positioned within a housing of the flexible elongate member 102. In that regard, the housing is a separate component secured to the flexible elongate member 102 in some instances. In other instances, the housing is integrally formed as a part of the flexible elongate member 102.

The intravascular device 100 also includes a connector 110 adjacent the proximal portion 106 of the device. In that regard, the connector 110 is spaced from the proximal end 107 of the flexible elongate member 102 by a distance 112. Generally, the distance 112 is between 0% and 50% of the total length of the flexible elongate member 102. While the total length of the flexible elongate member can be any length, in some embodiments the total length is between about 1300 mm and about 4000 mm, with some specific embodiments have a length of 1400 mm, 1900 mm, and 3000 mm. Accordingly, in some instances the connector 110 is positioned at the proximal end 107. In other instances, the connector 110 is spaced from the proximal end 107. For example, in some instances the connector 110 is spaced from the proximal end 107 between about 0 mm and about 1400 mm. In some specific embodiments, the connector 110 is spaced from the proximal end by a distance of 0 mm, 300 mm, and 1400 mm.

The connector 110 is configured to facilitate communication between the intravascular device 100 and another device. More specifically, in some embodiments the connector 110 is configured to facilitate communication of data obtained by the component 108 to another device, such as a computing device or processor. Accordingly, in some embodiments the connector 110 is an electrical connector. In such instances, the connector 110 provides an electrical connection to one or more electrical conductors that extend along the length of the flexible elongate member 102 and are electrically coupled to the component 108. In other embodiments, the connector 110 is an optical connector. In such instances, the connector 110 provides an optical connection to one or more optical communication pathways (e.g., fiber optic cable) that extend along the length of the flexible elongate member 102 and are optically coupled to the component 108. Further, in some embodiments the connector 110 provides both electrical and optical connections to both electrical conductor(s) and optical communication pathway(s) coupled to the component 108. In that regard, it should again be noted that component 108 is comprised of a plurality of elements in some instances. In some instances, the connector 110 is configured to provide a physical connection to another device, either directly or indirectly. In other instances, the connector 110 is configured to facilitate wireless communication between the intravascular device 100 and another device. Generally, any current or future developed wireless protocol(s) may be utilized. In yet other instances, the connector 110 facilitates both physical and wireless connection to another device.

As noted above, in some instances the connector 110 provides a connection between the component 108 of the intravascular device 100 and an external device. Accordingly, in some embodiments one or more electrical conductors, one or more optical pathways, and/or combinations thereof extend along the length of the flexible elongate member 102 between the connector 110 and the component 108 to facilitate communication between the connector 110 and the component 108. Generally, any number of electrical conductors, optical pathways, and/or combinations thereof can extend along the length of the flexible elongate member 102 between the connector 110 and the component 108. In some instances, between one and ten electrical conductors and/or optical pathways extend along the length of the flexible elongate member 102 between the connector 110 and the component 108. For the sake of clarity and simplicity, the embodiments of the present disclosure described below include three electrical conductors. However, it is understood that the total number of communication pathways and/or the number of electrical conductors and/or optical pathways is different in other embodiments. More specifically, the number of communication pathways and the number of electrical conductors and optical pathways extending along the length of the flexible elongate member 102 is determined by the desired functionality of the component 108 and the corresponding elements that define component 108 to provide such functionality.

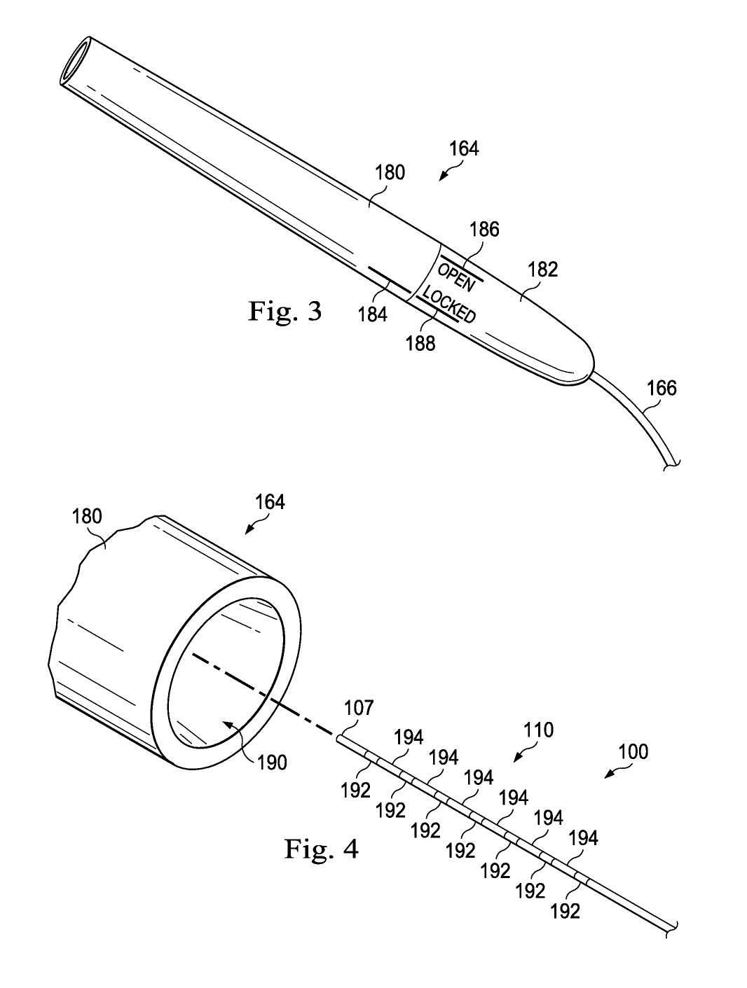

Referring now to FIG. 2, shown therein is a system 150 according to an embodiment of the present disclosure. As shown, the system 150 includes an instrument 152. In that regard, in some instances instrument 152 is an intravascular device as described above in the context of FIG. 1. Accordingly, in some instances the instrument 152 includes features similar to those discussed above with. In the illustrated embodiment, the instrument 152 is a guide wire having a distal portion 154 and a housing 156 positioned adjacent the distal portion. In that regard, the housing 156 is spaced approximately 3 cm from a distal tip of the instrument 152. The housing 156 is configured to house one or more sensors, transducers, and/or other monitoring elements configured to obtain the diagnostic information about the vessel. In the illustrated embodiment, the housing 156 contains at least a pressure sensor configured to monitor a pressure within a lumen in which the instrument 152 is positioned. A shaft 158 extends proximally from the housing 156. A torque device 160 is positioned over and coupled to a proximal portion of the shaft 158. A proximal end portion 162 of the instrument 152 is coupled to a connector 164. Connector 164 will be described in greater detail below with reference to at least FIGS. 3-10. A cable 166 extends from connector 164 to a connector 168. In some instances, connector 168 is configured to be plugged into an interface 170. In that regard, interface 170 is a patient interface module (PIM) in some instances. In some instances, the cable 166 is replaced with a wireless connection. In that regard, it is understood that various communication pathways between the instrument 152 and the interface 170 may be utilized, including physical connections (including electrical, optical, and/or fluid connections), wireless connections, and/or combinations thereof.

The interface 170 is communicatively coupled to a computing device 172 via a connection 174. Computing device 172 is generally representative of any device suitable for performing the processing and analysis techniques discussed within the present disclosure and, in particular, the processing and analysis techniques for the intravascular devices described in the context of FIG. 1. In some embodiments, the computing device 172 includes a processor, random access memory, and a storage medium. In that regard, in some particular instances the computing device 172 is programmed to execute steps associated with the data acquisition and analysis described herein. Accordingly, it is understood that any steps related to data acquisition, data processing, instrument control, and/or other processing or control aspects of the present disclosure may be implemented by the computing device using corresponding instructions stored on or in a non-transitory computer readable medium accessible by the computing device. In some instances, the computing device 172 is a console device. In some particular instances, the computing device 172 is similar to the s5™ Imaging System or the s5i™ Imaging System, each available from Volcano Corporation. In some instances, the computing device 172 is portable (e.g., handheld, on a rolling cart, etc.). Further, it is understood that in some instances the computing device 172 comprises a plurality of computing devices. In that regard, it is particularly understood that the different processing and/or control aspects of the present disclosure may be implemented separately or within predefined groupings using a plurality of computing devices. Any divisions and/or combinations of the processing and/or control aspects across multiple computing devices are within the scope of the present disclosure.

Together, connector 164, cable 166, connector 168, interface 170, and connection 174 facilitate communication between the one or more sensors, transducers, and/or other monitoring elements of the instrument 152 and the computing device 172. However, this communication pathway is exemplary in nature and should not be considered limiting in any way. In that regard, it is understood that any communication pathway between the instrument 152 and the computing device 172 may be utilized, including physical connections (including electrical, optical, and/or fluid connections), wireless connections, and/or combinations thereof. In that regard, it is understood that the connection 174 is wireless in some instances. In some instances, the connection 174 includes a communication link over a network (e.g., intranet, internet, telecommunications network, and/or other network). In that regard, it is understood that the computing device 172 is positioned remote from an operating area where the instrument 152 is being used in some instances. Having the connection 174 include a connection over a network can facilitate communication between the instrument 152 and the remote computing device 172 regardless of whether the computing device is in an adjacent room, an adjacent building, or in a different state/country. Further, it is understood that the communication pathway between the instrument 152 and the computing device 172 is a secure connection in some instances. Further still, it is understood that, in some instances, the data communicated over one or more portions of the communication pathway between the instrument 152 and the computing device 172 is encrypted.

It is understood that one or more components of the system 150 are not included, are implemented in a different arrangement/order, and/or are replaced with an alternative device/mechanism in other embodiments of the present disclosure. For example, in some instances, the system 150 does not include interface 170. In such instances, the connector 168 (or other similar connector in communication with instrument 152) may plug into a port associated with computing device 172. Alternatively, the instrument 152 may communicate wirelessly with the computing device 172. Generally speaking, the communication pathway between the instrument 152 and the computing device 172 may have no intermediate nodes (i.e., a direct connection), one intermediate node between the instrument and the computing device, or a plurality of intermediate nodes between the instrument and the computing device.

Referring now to FIG. 3, shown therein is a diagrammatic perspective view of the connector 164. As shown the connector 164 is defined by a housing having a body portion 180 and a nose portion 182. In the illustrated embodiment, the nose portion 182 is rotatable relative to the body portion 180 between an open position and a closed or locked position. In that regard, as will be discussed below, in the open position the connector 164 is configured to receive a connector portion of an intravascular device and in the closed position the connector 164 engages the received connector portion of the intravascular device. To this end, the connector 164 includes visual indicators that inform a user as to the current position of the connector relative to the open and closed position. In particular, the body portion 180 includes an indicator 184, while the nose portion 182 includes indicators 186 and 188. In the illustrated embodiment, the indicators 184, 186, and 188 take the form of line markings. However, it is understood that the indicators can take any suitable visible form. As shown, when the indicator 184 is aligned with the indicator 186, the connector 164 is in the open position. When the indicator 184 is aligned with the indicator 188, the connector 164 is in the closed position. It is also understood that in some embodiments, the indicators are active indicators that emit a signal (e.g., light or sound) based on the relative positions of the body portion 180 and the nose portion 182 and/or changes to the relative positions. For example, a first color of light may be emitted when the connector is in the open position and a second color of light may be emitted when the connector is in the closed position. Similarly, an audible sound may indicate movement of the connector between the open and closed positions and/or a proper engagement between a connection portion of an intravascular device and the connector. In some embodiments, the body portion 180 and/or the nose portion 182 is formed of a transparent or translucent material such that a proximal section of an intravascular device received within the connector 164 may be visualized by a user.

Referring now to FIG. 4, shown therein is a diagrammatic perspective view of the connector 164 and the proximal connector 110 of the intravascular device 100 according to an embodiment of the present disclosure. As shown, the body portion 180 of the connector 164 includes an opening 190 at its distal end that is sized and shaped to receive the proximal end of the intravascular device 100, including proximal connector 110. In the illustrated embodiment, the proximal connector 110 includes a plurality of conductive elements 192 separated by a plurality of insulating spacers 194. It is understood, however, that the proximal connector 110 may have any number of conductive elements or electrical contacts, including one, two, three, four, five, six, seven, eight, nine, ten, and so on. However, for most applications the proximal connector 110 will have between two and seven conductive elements.

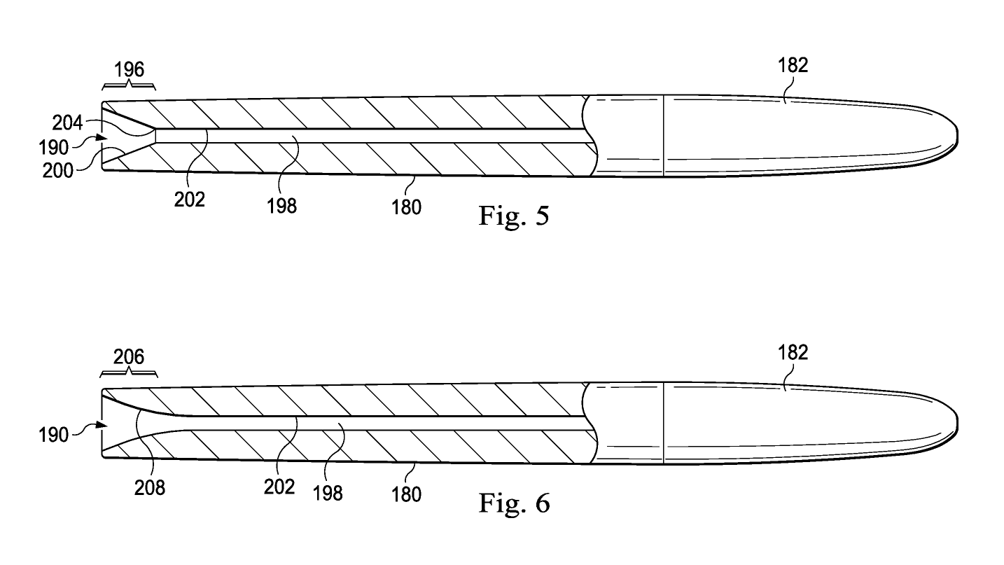

Referring now to FIG. 5, shown therein is a diagrammatic, partial cross-sectional side view of the connector 164 according to an embodiment of the present disclosure. As shown in FIG. 5, the opening 190 of the connector 164 has a tapered section 196 that leads to a central lumen 198 having a substantially constant diameter. A conical surface 200 defines the tapered section 196 of the opening 190, while a cylindrical surface 202 defines the central lumen 198. The conical surface 200 serves to guide the proximal end 107 of the intravascular device 100 into the central lumen 198. In that regard, the larger diameter of the opening 190 adjacent the outer, distal boundary of the body 180 facilitates easier insertion of the proximal end 107 of the intravascular device 100 into the connector, while the tapered section serves to guide the distal end 107 to the smaller central lumen 198. As a result, the need for a user to precisely place the proximal end 107 of the intravascular device 100 directly into an opening the size of the smaller central lumen 198 is eliminated. In the illustrated embodiment of FIG. 5, a transition point or corner 204 is created at the junction of the conical surface 200 with the cylindrical surface 202. In some instances, it is desirable to eliminate the transition point or corner 204 in order to prevent potential damage to the fragile proximal portion of the intravascular device 100 that will be received within the connector 164.

Referring now to FIG. 6, shown therein is a diagrammatic, partial cross-sectional side view of the connector 164 having an alternative implementation of opening 190 that eliminates the transition point or corner 204 of the embodiment of FIG. 5. In particular, as shown in FIG. 6 the opening 190 has an arcuately tapered section 206 that leads to the central lumen 198. In that regard, an arcuately tapering surface 208 extends from the outer, distal boundary of the body portion 180 to the cylindrical surface 202. The arcuately tapering surface 208 curves in such a manner that no points, corners, or other sharp transitions are created. Instead, a smooth arcuate transition is created from the larger diameter of the opening 190 at the outer, distal boundary of the body portion 180 to the central lumen 198. As a result, the potential for damage to the proximal portion of the intravascular device 100 is significantly decreased.

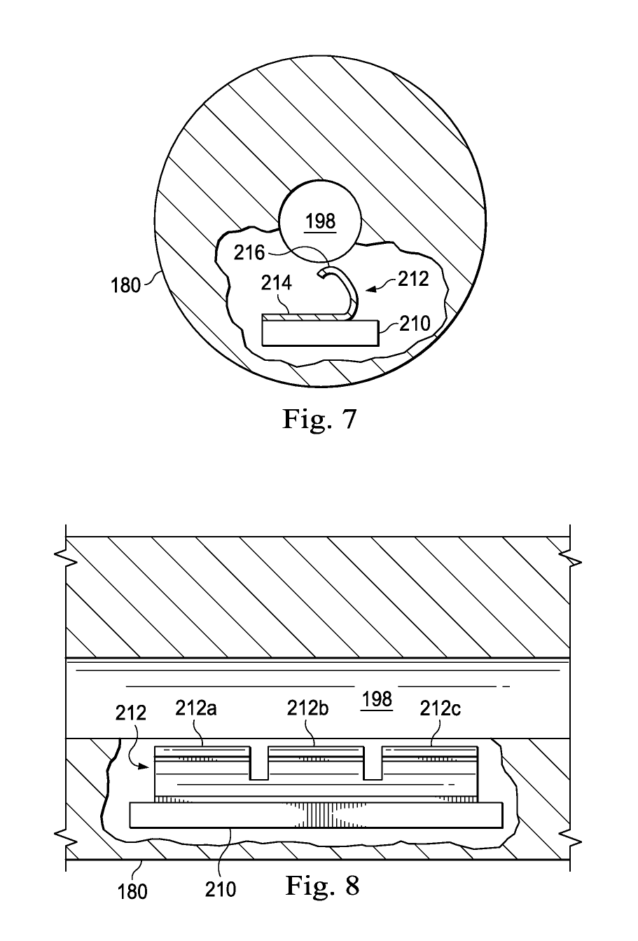

Referring now to FIGS. 7 and 8, the connector 164 is illustrated in an open position. In particular, FIG. 7 provides a diagrammatic cutaway end view of the connector in the open position, while FIG. 8 provides a diagrammatic cutaway side view of the connector in the open position. Various components and features of the connector 164 are not shown in FIGS. 7 and 8 in order to illustrate the general functionality of the connector 164. It is understood that this is for clarity and no limitation to the design of the connector 164 is intended thereby.

As shown, the connector 164 includes a mounting structure 210 upon which one or more electrical contacts are mounted. In the illustrated embodiment, an electrical contact 212 having contact portions 212a, 212b, and 212c is mounted to the mounting structure. Generally, any number of electrical contacts may be secured to the mounting structure, including one, two, three, four, five, six, seven, eight, nine, ten, and so on. However, for most applications between two and seven electrical contacts are used. In some instances, the number of electrical contacts is based upon the number of electrical connectors included on the intravascular device(s) that the connector 164 is to be used with. In that regard, the connector 164 may include the same number of electrical contacts as the intravascular device has electrical connectors. Alternatively, the connector 164 may include more electrical contacts than the intravascular device has electrical connectors or less electrical contacts than the intravascular device has electrical connectors. As shown, the electrical contact 212 includes a portion 214 that is fixedly secured to the mounting structure 210 and a portion 216 that extends up and away from the mounting structure 210. Portion 214 may be fixedly secured to the mounting structure 210 using any suitable techniques for the materials used, including without limitation soldering, welding, gluing, mechanical coupling, and/or otherwise securing the components together. In the illustrated embodiment, the portion 216 is generally arcuate. In particular, portion 216 is curved such that a free end of the electrical contact is directed back down toward portion 214 and a rounded section of portion 216 is configured to engage an intravascular device received within the central lumen 198.

In some implementations, the mounting structure 210 is a printed circuit board. In that regard, the one or more electrical contacts mounted to the printed circuit board are electrically coupled to one or more leads or conductive pathways of the printed circuit board in some instances. Further, the cable 166 extending from the connector 164 is in electrical communication with the one or more leads or conductive pathways of the printed circuit board. As a result, when an intravascular device is electrically coupled to the one or more electrical contacts mounted to the printed circuit board, signals can be communicated between the interface 170 or processing system 172 and the intravascular device 100. In some instances, a distal portion of conductors extending through the cable 166 are electrically coupled to the circuit board. In other instances, one or more conductive paths and/or conductors bridge the gap between the printed circuit board and the conductors of the cable 166. For example, in some instances the cable 166 includes a plug that is configured to engage a socket of the connector 164. In such instances, the plug and socket may use a standardized connection format (e.g., USB, FireWire, and/or other standard data and/or power interfaces) and/or a custom connection format.

As shown in FIGS. 7 and 8, when the connector 164 is in the open position the electrical contact 212 is positioned adjacent to, but out of the central lumen 198. As a result, the proximal end 107 of the intravascular device 100 is free to pass through the central lumen 198 without friction or engagement with the electrical contact(s) of the connector 164. This again results in the potential for damage to the proximal portion of the intravascular device 100 being significantly decreased. In some implementations, one or more projections or surface features are formed on a proximal section of the cylindrical surface 202 defining the central lumen 198 in order to provide tactile feedback to the user that the proximal end 107 of the intravascular device 100 is approaching the proximal end of the central lumen 198 in an effort to prevent a user from accidentally ramming the proximal end 107 of the intravascular device 100 into the wall bounding the proximal end of the central lumen 198, which could result in damage to the intravascular device 100. In that regard, when the proximal end 107 of the intravascular device 100 reaches the one or more projections or surface features, the increase in friction or resistance associated with advancing the intravascular device 100 further into the central lumen will provide tactile feedback to the user to proceed with caution.

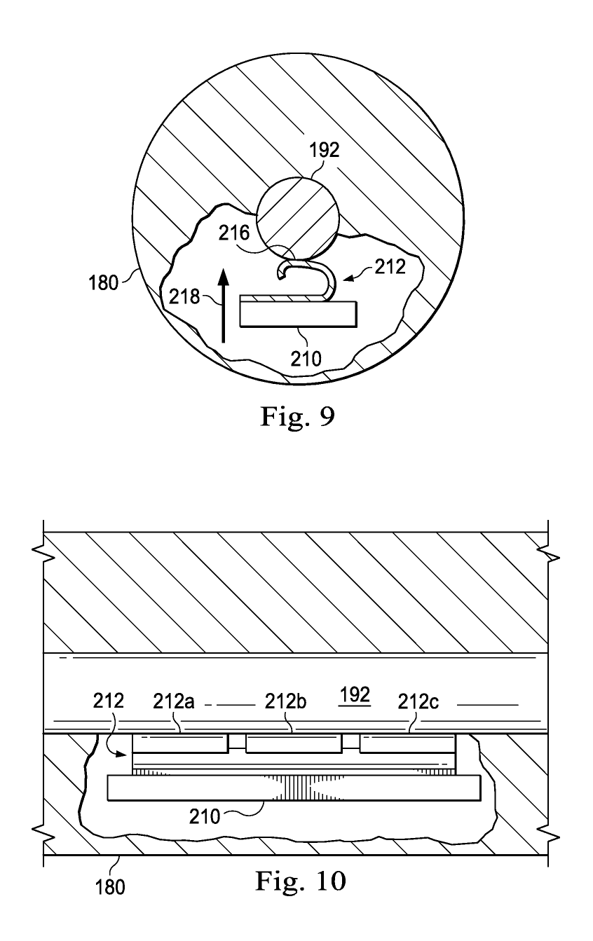

Referring now to FIGS. 9 and 10, the connector 164 is illustrated in a closed position. In particular, FIG. 9 provides a diagrammatic cutaway end view of the connector in the closed position, while FIG. 10 provides a diagrammatic cutaway side view of the connector in the closed position. Again, various components and features of the connector 164 are not shown in FIGS. 9 and 10 in order to illustrate the general functionality of the connector 164. It is understood that this is for clarity and no limitation to the design of the connector 164 is intended thereby. As shown in FIGS. 9 and 10, when the proximal end 107 of the intravascular device 100 is positioned within the central lumen 198 of the connector 164 and the connector is moved to the closed position, the electrical contact 212 engages a conductive element 192 of the intravascular device. In that regard, when the connector 164 is moved from the open position to the closed position, the mounting structure 210 moves in the direction of arrow 218 towards the central lumen 198. In that regard, while arrow 218 is indicated as being linear, it is understood that in some embodiments where rotation of the nose portion 182 relative to the body portion 180 causes the mounting structure 210 to move toward the central lumen 198, the mounting structure 210 will move along an arcuate path as it moves closer to the central lumen 198. For example, in some instances the mounting structure 210 is coupled to a cam structure such that rotation of the nose portion 182 relative to the body portion 180 causes the mounting structure 210 to move in accordance with the profile of the cam structure towards the central lumen 198 (or away from the central lumen 198 when moving from the closed position to the open position). In the closed position, the portion 216 of the electrical contact 212 engages the conductive element 192 of the intravascular device 100. In that regard, it is understood that at least a section of the portion 216 will extend within the central lumen 198 to engage the conductive element 192. Accordingly, in some instances, the electrical contact 212 is positioned adjacent to the central lumen 198 in the open position and extends at least partially across the central lumen 198 in the closed position to be electrically coupled a conductive element of the intravascular device positioned within the central lumen.

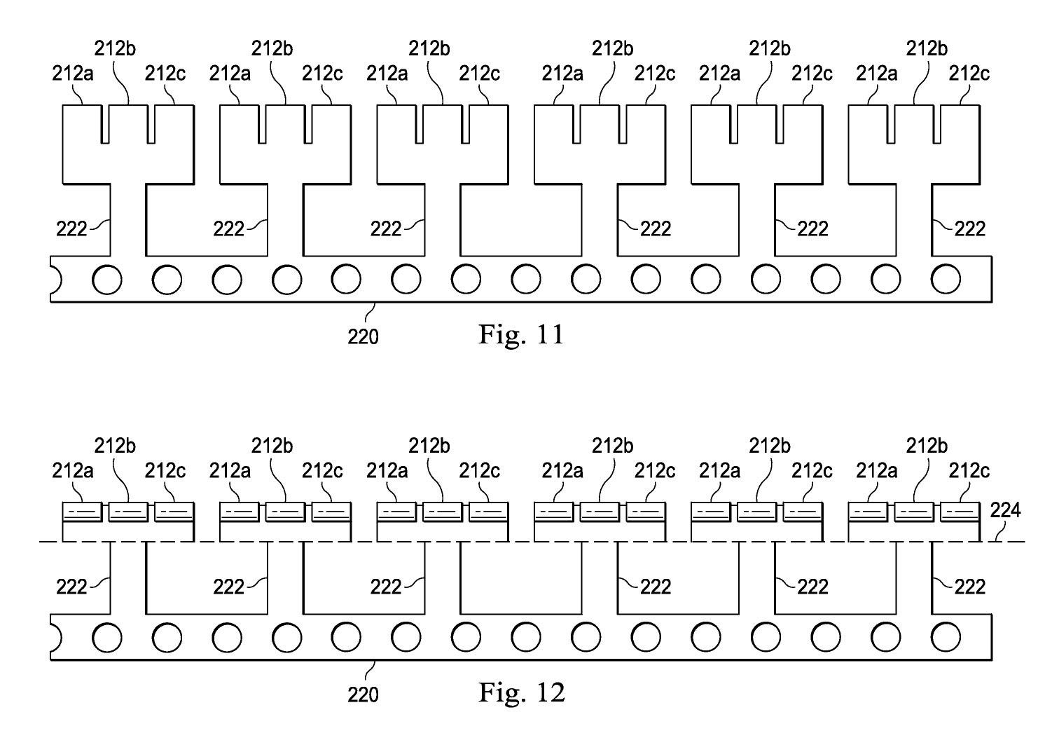

Referring now to FIGS. 11 and 12, aspects of manufacturing electrical contact 212 will be described according to an embodiment of the present disclosure. Referring initially to FIG. 11, a plurality of electrical contacts 212, each having contact portions 212a, 212b, and 212c, are shown in a planar or flattened format. Each of the contacts 212 is connected to a lead frame 220 by leads 222. In some implementations, the electrical contacts 212 and the leads 222 are spaced apart along the length of the lead frame 220 by distance(s) corresponding to the desired spacing for the electrical contacts 212 to be coupled to a mounting structure, such as a printed circuit board, as a group (i.e., without requiring separation and/or individual placement of each of the electrical contacts 212 onto the mounting structure). In that regard, the electrical contacts 212 and associated leads 222 are equally spaced along the length of the lead frame 220 in the illustrated embodiment. However, in other embodiments the spacing between electrical contacts 212 varies along the length of the lead frame 220. Generally, the electrical contacts 212 and associated leads 222 may be spaced by any desirable distance. In some instances, the number of electrical contacts 212 mounted to the lead frame 220 is equal to the number of electrical contacts 212 to be mounted to the mounting structure. In other instances, more or less electrical contacts 212 are coupled to the lead frame 220 than will be mounted to the mounting structure. Electrical contacts can be removed from the lead frame 220 and/or additional electrical contacts can be supplied (e.g., from other lead frames) when the number of electrical contacts is not equal to the number to be mounted to the mounting structure.

Referring now to FIG. 12, the contact portions 212a, 212b, and 212c of each of the plurality of electrical contacts 212 have been rolled or otherwise bent into the configuration shown in FIGS. 6 and 7. Any suitable technique can be utilized to transition the contact portions 212a, 212b, and 212c from the planar or flattened configuration of FIG. 11 into the arcuate configuration of FIGS. 6, 7, and 12. For example, in some instances the contact portions 212a, 212b, and 212c are bent. With the contact portions 212a, 212b, and 212c shaped, the electrical contacts 212 can be mounted to the mounting structure. As noted above, in some instances the electrical contacts 212 are mounted to the mounting structure while still coupled to the lead frame 220 by leads 222. Accordingly, in some instances, after coupling the electrical contacts 212 to the mounting structure the electrical contacts 212 are separated from the lead frame 220. In the illustrated embodiment of FIG. 12, a line 224 indicates a separation point for the electrical contacts 212 from the leads 222 and the lead frame 220. In other instances, the line 224 is positioned closer to the lead frame 220 such that at least portion of the leads 222 remain coupled to the electrical connectors 212. The electrical connectors 212 may be separated from the lead frame 220 and/or leads 222 using any suitable technique.

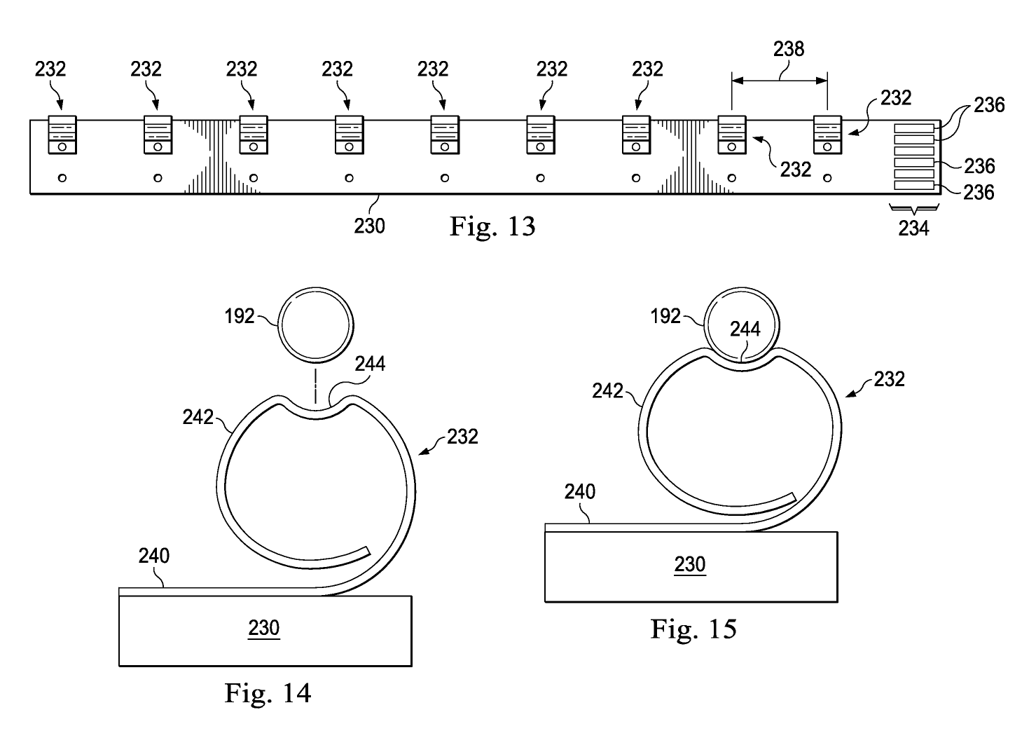

Referring now to FIG. 13, shown therein is a mounting structure 230 with a plurality of electrical contacts 232 secured thereto according to an embodiment of the present disclosure. In the illustrated embodiment, the mounting structure 230 is a printed circuit board and the electrical contacts 232 are electrically coupled to lead lines and/or other conductive portions of the printed circuit board. In that regard, an electrical connector 234 having a plurality of electrical pads or contacts 236 is defined adjacent an end of the mounting structure 230. In some instances, the electrical connector 234 and associated contact pads 236 are configured to interface, either directly or indirectly, with conductors of cable 166 and/or other communication cable. In the illustrated embodiment, the electrical contacts 232 are spaced apart by a distance 238.

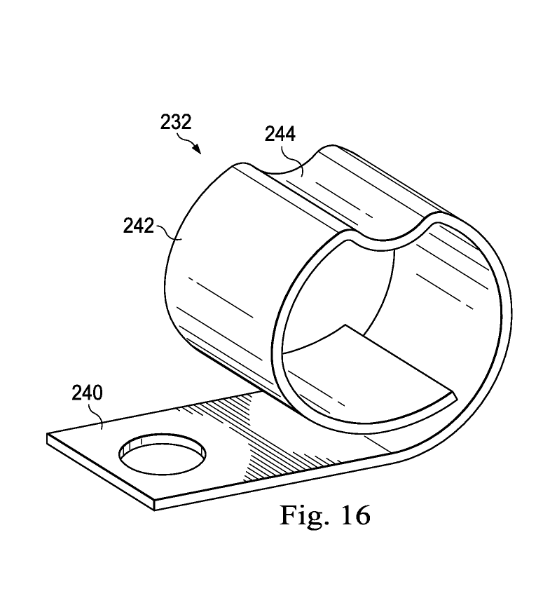

Referring now to FIGS. 14-16, additional features of the electrical contacts 232 will be described. In that regard, FIG. 14 shows the mounting structure 230 and associated electrical contact 232 in an open position, spaced from a connection portion of an intravascular device, while FIG. 15 shows the mounting structure 230 and associated electrical contact 232 in a closed position, engaged with the connection portion of the intravascular device. FIG. 16 provides a diagrammatic perspective view of the electrical contact 232. As shown, the electrical contact 232 includes a portion 240 that is fixedly secured to the mounting structure 230 and a portion 242 that extends up and away from the mounting structure 230. Portion 240 may be fixedly secured to the mounting structure 230 using any suitable techniques for the materials used, including without limitation soldering, welding, gluing, mechanical coupling, and/or otherwise securing the components together. In the illustrated embodiment, the portion 242 is generally arcuate, but with a recess 244 defined therein. In particular, portion 242 is curved such that a free end of the electrical contact 232 is directed back down toward portion 240 and a rounded section of portion 242 includes the recess 244 that is sized and shaped to receivingly engage an electrical connector of an intravascular device. In some instances, the recess 244 is arcuate with a radius of curvature sized to mate with the electrical connector of an intravascular device. Accordingly, in some implementations the recess 244 has a radius of curvature equal to or slightly larger than the radius of curvature of the electrical connector it is to interface with. To this end, FIG. 14 shows the electrical contact 232 spaced from the electrical connector 192, while FIG. 15 shows the electrical contact 232 engaged with the electrical connector 192. As shown, when in the closed or engaged position, the electrical connector 192 is received within the recess 244. In some instances, the recess 244 of electrical contact 232 defines a larger contact surface than that provided by contact 212 describe previously. In this manner, the recess 244 can improve the mating engagement of the electrical contact 232 to the electrical connector 192 and, therefore, improve the electrical connection between the components.

Persons skilled in the art will also recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An intravascular system, comprising:
an intravascular device comprising:
a flexible elongate member having a proximal portion and a distal portion;
at least one electronic component secured to the distal portion of the flexible elongate member; and
at least one electrical connector secured to the proximal portion of the flexible elongate member, wherein the at least one electrical connector is electrically coupled to the at least one electronic component secured to the distal portion of the flexible elongate member; and
a connector having at least one electrical contact configured to selectively interface with the at least one electrical connector of the intravascular device, the connector including:
a housing defining a central lumen,
a mounting structure positioned within the housing adjacent the central lumen,
wherein the at least one electrical contact is secured to the mounting structure;
wherein the mounting structure is movable relative to the central lumen between an open position and a closed position;
wherein in the open position the at least one electrical contact is positioned adjacent to the central lumen of the housing such that the at least one electrical connector of the intravascular device can be inserted into the central lumen;
wherein in the closed position the at least one electrical contact extends at least partially across the central lumen of the housing to electrically couple to the at least one electrical connector of the intravascular device positioned within the central lumen.

2. The system of claim 1, wherein movement of a first portion of the housing relative to a second portion of the housing moves the mounting structure between the open position and the closed position.

3. The system of claim 2, wherein rotation of a first portion of the housing relative to a second portion of the housing moves the mounting structure between the open position and the closed position.

4. The system of claim 3, wherein a ninety degree rotation of a first portion of the housing relative to a second portion of the housing moves the mounting structure between the open position and the closed position.

5. The system of claim 1, wherein the at least one electrical contact includes a recess sized and shaped to the at least one electrical connector of the intravascular device.

6. The system of claim 5, wherein engagement of the at least one electrical connector with the recess of the at least one electrical contact provides a tactile feedback to a user indicating a secure engagement of the at least one electrical contact with the at least one electrical connector.

7. The system of claim 1, wherein the at least one electronic component is at least one of a pressure sensing component, an intravascular imaging component, and a flow sensing component.

8. The system of claim 1, wherein the mounting structure is a printed circuit board.

9. The system of claim 1, wherein the housing is formed of a transparent or translucent material such that the at least one electrical connector of the intravascular device may be visualized by a user when positioned within the central lumen of the housing.

10. A method, comprising:
providing a connector having:
a housing with a central lumen,
a mounting structure, and
at least one electrical contact secured to the mounting structure;
moving the connector to an open position such that the at least one electrical contact is positioned adjacent to the central lumen of the housing such that a connection portion of an intravascular device can be inserted into the central lumen of the housing;
inserting the connection portion of the intravascular device into the central lumen of the housing while the connector is in the open position; and
moving the connector to a closed position such that the at least one electrical contact extends at least partially across the central lumen of the housing to electrically couple the at least one electrical contact of the connector to at least one electrical connector of the connection portion of the intravascular device received within the central lumen of the housing.

11. The method of claim 10, wherein the at least one electrical connector is electrically connected to an electronic component positioned at a distal portion of the intravascular device such that the at least one electrical contact is electrically coupled to the electronic component when the at least one electrical contact is electrically coupled to the at least one electrical connector of the connection portion of the intravascular device.

18. The method of claim 11, wherein the electronic component is at least one of a pressure sensing component, an intravascular imaging component, and a flow sensing component.

19. A connector for an intravascular system, comprising:
a housing defining a central lumen;
a mounting structure positioned within the housing adjacent the central lumen; and
at least one electrical contact secured to the mounting structure;
wherein the mounting structure is movable relative to the central lumen between an open position and a closed position;
wherein in the open position the at least one electrical contact is positioned adjacent to the central lumen of the housing such that at least one connector of an intravascular device can be inserted into the central lumen;
wherein in the closed position the at least one electrical contact extends at least partially across the central lumen of the housing to electrically couple to the at least one electrical connector of the intravascular device positioned within the central lumen.

20. The connector of claim 19, wherein the housing includes an opening in communication with the lumen, the opening having an arcuately tapering profile from an outer boundary of the housing into the lumen.
